(19) United States
(12) Patent Application Publication
(10) Pub. No.: US 2020/0048709 A1
(43) Pub. Date: Feb. 13, 2020

(54) AUTOMATED DIFFERENTIAL EXPRESSION ANALYSIS OF RNA SEQUENCING DATA

(71) Applicant: ExxonMobil Research and Engineering Company, Annandale, NJ (US)

(72) Inventors: Antonio R. Paiva, Clinton, NJ (US); Giovanni Pilloni, Jersey City, NJ (US)

(21) Appl. No.: 16/533,857

(22) Filed: Aug. 7, 2019

Related U.S. Application Data
(60) Provisional application No. 62/717,564, filed on Aug. 10, 2018.

(51) Int. Cl. C12Q 1/6876; C12Q 1/6869; G16B 20/00; G16B 30/20; G16B 30/10; G16B 40/10; G16B 50/30

(52) U.S. Cl. CPC C12Q 1/6876; C12Q 1/6869; G16B 20/00; G16B 50/30; G16B 30/10; G16B 40/10; G16B 30/20

(57) ABSTRACT

Methods and systems for differential expression analysis of ribonucleic acid sequencing (RNA-Seq) data, and in particular to an automated workflow for differential expression analysis of RNA-Seq data. More particularly, an automated workflow for differential expression analysis of RNA-Seq data permitting analysis of pairs of any number of RNA-Seq reads subjected to multiple experimental conditions and tested in replicate.

### CROSS-REFERENCE TO RELATED APPLICATIONS

This application claims the benefit of U.S. Provisional Application No. 62/717,564, filed on Aug. 10, 2018, the entire contents of which are incorporated herein by reference.

### FIELD

The present disclosure relates to differential expression analysis of ribonucleic acid sequencing (RNA-Seq) data, and in particular to an automated workflow for differential expression analysis of RNA-Seq data.

### BACKGROUND

RNA-Seq data may be used to identify, analyze, and quantify the expression of a particular gene at a certain moment in time and under certain experimental conditions. RNA-Seq utilizes one or more next generation sequencing platforms, allowing rapid analysis of various sized genomes compared to previous sequencing technologies. Typically, RNA-Seq consists of some or all of identifying a biological sample of interest that has been subjected to one or more experimental conditions, isolating RNA therefrom, obtaining RNA reads, aligning the RNA reads to a transcriptome (e.g., of a transcriptome library), and performing various downstream analyses, such as differential expression analysis.

Differential expression analysis using RNA-Seq data is a methodology employed to evaluate how biological organisms (e.g., microorganisms and other biological organisms, including any prokaryotes and eukaryotes) respond to changes in conditions. For example, such analysis may be used for evaluating how a microorganism responds to changes in concentration of a given compound within its environment by exposing the same microorganism to various concentrations of the compound, with all other variables remaining constant or controlled. Each selected concentration may additionally be tested in replicates (e.g., duplicates, triplicates, and the like) to control for natural variability in the testing and the microorganisms themselves. In response to the experimental conditions, the microorganisms may respond by transcribing different genes or different gene levels (i.e., intensity or quantity), which results in different proteins or protein levels operating in the microorganism. Accordingly, RNA-Seq data comprised of sequenced RNA reads (i.e., messenger RNA (mRNA) transcripts or reverse transcribed cDNA) may be used to identify which gene(s) and how much of said gene(s) is expressed in the presence of a given condition by differential expression analysis.

Differential expression analysis of RNA-Seq data is typically a time-consuming and computationally intensive process. Various tools may be employed to perform the analysis to identify differentially expressed genes between one or more experimental conditions. These tools typically stand-alone and facilitate one of mapping RNA reads to a reference transcriptome, determining transcript quantity, and differential gene expression analysis (e.g., using statistical methodology). Use of such tools typically requires substantial user familiarity with each tool and is generally tedious and slow, requiring substantial computing power to execute each of the steps for each of the tools. Moreover, such traditional tools are currently unable to simultaneously analyze large datasets having both multiple replicates and multiple conditions.

### SUMMARY

The present disclosure relates to differential expression analysis of ribonucleic acid sequencing (RNA-Seq) data, and in particular to an automated workflow for differential expression analysis of RNA-Seq data. More particularly, the automated workflow for differential expression analysis of RNA-Seq data described herein permits analysis of any number of RNA-Seq reads corresponding to multiple experimental conditions, as well as any potential replicates tested thereof.

In one or more aspects, the present disclosure provides a method for performing automated differential expression analysis using an RNA-Seq workflow. The method comprises using at least one data processing unit having at least one processor and memory coupled to the at least one processor. The memory is operative to store instructions that, when executed by the processor, cause the processor to perform the automated differential expression analysis using the RNA-Seq workflow. The instructions include identifying a plurality of RNA-Seq reads from genomic samples, the plurality of genomic samples having been subjected to at least one experimental condition; aligning the plurality of RNA-Seq reads to a transcriptome for the genomic samples; quantifying gene expression for the plurality of RNA-Seq reads; and quantifying differential gene expression in the plurality of RNA-Seq reads between a combination pair of the experimental condition.

In one or more aspects, the present disclosure provides a method for performing automated differential expression analysis using an RNA-Seq workflow. The method comprises receiving a user input defining the RNA-Seq workflow, the workflow having one or more user specified instructions. The method further includes using at least one data processing unit having at least one processor and memory coupled to the at least one processor. The memory is operative to store instructions that, when executed by the processor, cause the processor to perform the automated differential expression analysis using the RNA-Seq workflow. The instructions include identifying a plurality of RNA-Seq reads from genomic samples, the plurality of genomic samples having been subjected to at least one experimental condition; aligning the plurality of RNA-Seq reads to a transcriptome for the genomic samples; quantifying gene expression for the plurality of RNA-Seq reads; and quantifying differential gene expression in the plurality of RNA-Seq reads between a combination pair of the experimental condition.

In one or more aspects, the present disclosure provides a system for performing automated differential expression analysis using an RNA-Seq workflow. The system includes at least one data processing unit comprising at least one processor and memory coupled to the at least one processor. The memory is operative to store instructions that, when executed by the processor, cause the processor to perform the automated differential expression analysis using the RNA-Seq workflow. The instructions identify a plurality of RNA-Seq reads from genomic samples, the plurality of genomic samples having been subjected to at least one experimental condition; align the plurality of RNA-Seq reads to a transcriptome for the genomic samples; quantify gene expression for the plurality of RNA-Seq reads; and quantifying differential gene expression in the plurality of RNA-Seq reads between a combination pair of the experimental condition.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a schematic flowchart demonstrating one or more aspects of the parallel analysis and combination pairing of the RNA-Seq differential expression analysis workflows of the present disclosure.

FIG. 2 is a schematic flowchart demonstrating one or more aspects of the parallel analysis and combination pairing of the RNA-Seq differential expression analysis workflows of the present disclosure.

FIG. 3 is a chart showing lactate, acetate, and sulfate concentrations in Desulfovibrio vulgaris Hildenbourough growing with different indole concentrations, as described in Example 2.

FIG. 4 is a chart showing differential expression analysis performed according to the automated differential expression analysis workflow of the present disclosure and compared to a differential expression analysis performed commercially by a third-party vendor.

### DETAILED DESCRIPTION

The present disclosure relates to differential expression analysis of ribonucleic acid sequencing (RNA-Seq) data, and in particular to an automated workflow for differential expression analysis of RNA-Seq data. More particularly, the automated workflow for differential expression analysis of RNA-Seq data described herein permits analysis of pairs of any number of RNA-Seq reads subjected to multiple experimental conditions, as well as any potential replicates tested thereof.

The RNA-Seq differential expression analysis workflows (also referred to herein simply as “RNA-Seq workflow(s)”) disclosed herein allow rapid differential gene expression analysis of large RNA-Seq read datasets, automatically adapting to the dataset including any number of input files (e.g., files comprising specific RNA-Seq reads), any number of replicates, and any number and type of conditions. The workflows are parallelized, such that certain operations are independently performed and, thereafter, a combination pair of experimental conditions is subsequently used in parallel for differential expression analysis. Accordingly, unlike traditional RNA-Seq differential expression analysis workflows, every combination pair of RNA-Seq reads may be analyzed for differential expression, thereby permitting a more robust review of the gene expression and the biological organisms' reaction to one or more experimental conditions, as described in greater detail below. Moreover, the workflow of the present disclosure may be run automatically with minimal user input, whereas traditional RNA-Seq differential expression analysis workflows require each operation (e.g., alignment, quantification, and the like) to be performed manually in sequence (i.e., from a command line or potentially a graphical user interface). Accordingly, the RNA-Seq differential expression analysis workflows described herein are streamlined and permit rapid and effective (e.g., without error) differential expression analysis across all experimental condition pairs of large datasets. The workflow automatically identify and aggregate all relevant data for differential expression analysis and process it though the steps of the workflow.

Moreover, the RNA-Seq differential expression analysis workflows of the present disclosure are user-friendly and adaptable, such that minimal user input is required and the workflow can be easily adapted to the user's needs, including handling the large datasets aforementioned (e.g., multiple experimental conditions and/or replicates) and automatically adapting to changes in the datasets. Such changes may include, for example, changes stemming from user configuration options and/or changes in the specific input data (e.g., the RNA-Seq read data). Changes that stem from user configuration options may include, but are not limited to, changes to the selection of a particular tool/algorithm for analysis in the workflow (e.g., for alignment), changes to the computational options of such tools/algorithms (e.g., to assess sensitivity to changes of algorithms or parameters thereof), changes to the input data (e.g., user input RNA-Seq data), and the like, and any combination thereof. The dynamic quality of the workflows of the present disclosure automatically links the various tools/algorithms/parameters together to perform the RNA-Seq differential expression analysis. For example, the workflows described herein standardize the use of various tools (e.g., alignment tool, quantification tool, and the like, as described herein) such that each of the tools can be used together with ease (i.e., the tools are made compatible via the workflow). Changes to the input data for analysis by one or more workflows described herein may include, for example, selection of particular input files, RNA sequencing runs, experimental conditions, replicate numbers, and the like, and any combination thereof. The workflows described herein automatically and dynamically adapts to any such changes because the workflows are designed to recognize sets of organism files, conditions, and replicates based on a general nomenclature pattern, as described herein, and which may be designed and adapted based on user preference. Example nomenclature may include, for instance, regular expressions on files of interest (e.g., each example condition in a file of interest is prefixed with the term “condition_”). As such, the workflows described herein are able to identify all of the relevant files, such as the relevant experimental conditions, to be considered and the corresponding RNA-Seq read files and apply all relevant analytical tools (i.e., computing steps) selected for the particular workflow.

The workflows described herein may be modified by a user to select certain tools for performing certain tasks of the workflow with seamless integration. The workflows may be associated (i.e., in electrical communication) with a display, such as a user interface, such that a user can input information into the display. The user may input an initial specification profile for performing the workflow, such as by specifying the dataset for differential expression analysis and selection of the specific tools for execution of the workflow (e.g., to evaluate different methodologies for arriving at the differential expression analysis results). Thereafter, the workflows of the present disclosure, unlike traditional workflows, adaptively combine the selected tools to compute all necessary steps of the RNA-Seq differential expression analysis workflow for all combination pairs of experimental conditions. Accordingly, the workflows described herein can adaptively analyze datasets using various different analysis tools. Moreover, in some embodiments, intermediate results of prior run datasets may be reused in subsequent RNA-Seq differential expression analysis workflows. Such reuse may lead to reduced runtime and computing power needs, for example.

One or more illustrative embodiments incorporating the embodiments of the present disclosure are included and presented herein. Not all features of a physical implementation are necessarily described or shown in this application for the sake of clarity. It is understood that in the development of a physical embodiment incorporating the embodiments of the present disclosure, numerous implementation-specific decisions must be made to achieve the developer's goals, such as compliance with system-related, business-related, government-related, and other constraints, which vary by implementation and from time to time. While a developer's efforts might be time-consuming, such efforts would be, nevertheless, a routine undertaking for those of ordinary skill in the art and having benefit of this disclosure.

Unless otherwise indicated, all numbers expressing quantities of ingredients, properties such as physical properties, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present disclosure. At the very least, and not as an attempt to limit the application of the doctrine of equivalents to the scope of the claim, each numerical parameter should at least be construed in light of the number of reported significant digits and by applying ordinary rounding techniques. Where the term “less than about” or “more than about” is used herein, the quantity being modified includes said quantity, thereby encompassing values “equal to.” That is “less than about 3.5%” includes the value 3.5%, as used herein.

While compositions and methods are described herein in terms of “comprising” various components or steps, the compositions and methods can also “consist essentially of” or “consist of” the various components and steps.

Various terms as used herein are defined hereinbelow. To the extent a term used in a claim is not defined below, it should be given the broadest definition persons in the pertinent art have given that term as reflected in one or more printed publications or issued patents.

As used herein, the terms “RNA sequencing” or “RNA-Seq,” and grammatical variants thereof, refers to next-generation sequencing of RNA in a biological sample at a given time and subjected to one or more experimental conditions.

As used herein, the term “RNA-Seq read” or simply “read,” and grammatical variants thereof, refers to a fragment of RNA, or reverse transcribed cDNA derived therefrom, received from analysis of RNA molecules obtained from a biological sample. Such reads may be obtained and/or amplified using various techniques including sequencing, polymerase chain reaction (PCR) amplification, mass spectrometry, and the like, or any combination thereof. The reads comprised of RNA may include total RNA, poylA-selection RNA, rRNA, depleted RNA, mRNA, and the like. Moreover, the reads may be in the form of paired-end reads or single-end reads, without departing from the scope of the present disclosure.

As used herein, the terms “RNA-Seq differential expression analysis workflow,” “RNA-Seq data workflow,” “RNA-Seq workflow,” or simply “workflow,” and grammatical variants thereof, refers to a sequence of modifiable process steps through which an analysis of a plurality of RNA-Seq reads from at least one genome sample subjected to at least one experimental condition passes for ultimate quantification of differential gene expression, excluding preparation and sequencing of biological samples (i.e., the workflows determine differential gene expression based on an already-sequenced RNA-Seq read). The differential gene expression is determined among combination pairs of the experimental condition(s).

As used herein, the term “experimental conditions,” and grammatical variants thereof, refers to one or more dependent variables that may be controlled or otherwise altered to measure the value of an independent variable. As used herein, the term “experimental conditions” encompass both control and test conditions.

As used herein, the term “combination pair of experimental conditions” or simply “combination pair,” and grammatical variants thereof, refers to two different sets of RNA-Seq reads derived from related biological specimens (e.g., same microorganism or related genetic variants thereof, or same culture of microorganisms), and subjected to different experimental conditions, which are typically comparable. For example, microorganism A may be cultured in the presence of a concentration C1 of drug K and separately cultured in the presence of a concentration C2 of the same drug K (e.g., while the microorganism A is the same, two samples of A are cultured in C1 and C2 of drug K). Accordingly, the combination pair would be C1 and C2, and the differential expression analysis would be based on exposure to the concentrations of drug K.

As used herein, the term “gene expression” or “expression,” and grammatical variants thereof, refers to the biochemical process of determining which genes are actively transcribed into RNA (i.e., within cells of a biological sample) under certain conditions (e.g., upon exposure to certain conditions). The term “differential gene expression” or “differential expression,” and grammatical variants thereof, as used herein, refers to comparison in gene expression of a biological sample between at least a combination pair of experimental conditions (e.g., a change in concentration of a condition, a type of condition, and the like). The qualifier “gene,” as in “gene expression,” is not limiting. That is, the embodiments described herein with reference to gene expression are applicable to any other transcriptome sequence or subsequence of interest, including at the exon level, the gene level, and the like, and any combination thereof. Such particular transcriptomes may be saved as a file identified by the workflows described herein using particular file nomenclature, as described herein.

As used herein, the term “transcriptome,” and grammatical variants thereof, refers to a sequence of RNA molecules that may be transcribed from one or more genomes. The term “transcriptomics” refers to the study of such transcriptomes and their functions.

In an aspect of the present disclosure, methods and systems are provided for performing an automated differential expression analysis based on RNA-Seq data according to a streamlined and modifiable RNA-Seq workflow, as described herein.

RNA-Seq data may be obtained from biological samples of interest, such as one or more microorganisms or a sample comprising one or more microorganisms that has been subjected to one or more experimental conditions. For example, such experimental conditions may include exposure to certain concentrations or types of environmental or other external agents (e.g., drugs, nutrients, pathogens, temperature, pressure, and the like, and any combination thereof). Accordingly, multiple samples of a particular biological specimen may be exposed to many different experimental conditions in order to determine the effect of gene expression in each sample compared to at least another such sample (e.g., the “combination pairs” described herein). Moreover, any particular experimental condition may be tested in replicate to account for natural biological variation across such samples or other variation, such as testing and equipment variation.

In order to gain meaningful information regarding the effect of the experimental conditions on the biological samples and the biological specimen itself (e.g., the particular microorganism), the RNA-Seq read data from each experimental condition and each replicate must be processed and analyzed, which may be in the range of millions or more RNA-Seq reads for each condition and each replicate. As described hereinabove, traditional RNA-Seq tools may be incompatible or otherwise unable to simultaneously analyze combination pairs in such large datasets, requiring significant time and computational power and often leading to errors and/or waste (e.g., time, resources, and the like). The workflows of the present disclosure perform RNA-Seq read alignment to a transcriptome, quantification of expression (e.g., at the exon level, the gene level, and/or the like), and quantification of differential expression across multiple datasets having multiple experimental conditions and replicates, where such workflows are modifiable and able to identify each combination pair automatically.

Example features of the workflows of the present disclosure a rapid, effective, and adaptive differential expression analysis across multiple datasets having many experimental conditions and replicates include: modular use of tools, adaptive data specification, and transparent handling of computation.

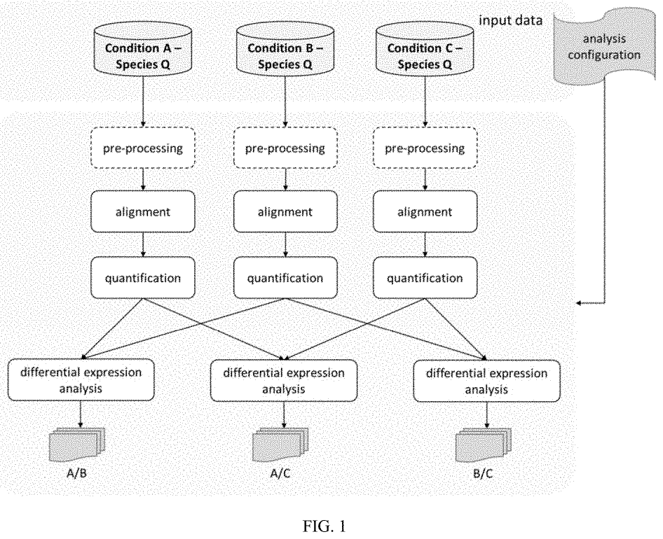

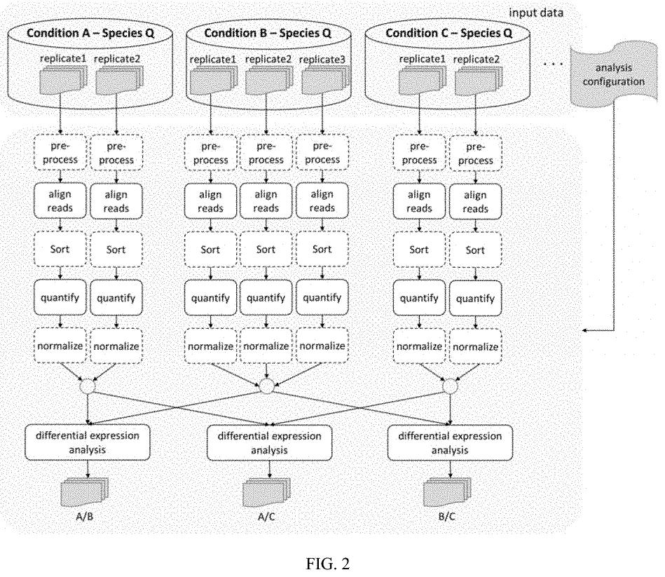

The RNA-Seq data workflows for performing automated differential expression analysis described herein allow modular use of various tools. Such tools are designed to perform one or more of the functions of at least alignment of RNA-Seq reads, quantification of gene expression, and differential expression quantification, among other functions described hereinbelow. These tools may be commercially available or otherwise available publically via open source software or code. For each analysis step, a user is able to select a specific tool from one or more available (e.g., supported) tools in the workflow. Accordingly, a user running the same RNA-Seq read dataset for differential expression analysis using the workflow of the present disclosure may seamlessly perform such analysis using one or a variety of available tools, thereby allowing the user to identify potential subtle differences in the analysis outcome, which may further influence scientific conclusions based on the RNA-Seq reads and experimental conditions. The default configuration setting, invocation format, and specific computational needs for each available tool is built into the workflow system, such that complications using each such tool are avoided (i.e., abstracted from the user). Such potential complications that are avoided due to the standardization of the workflows described herein may include, but are not limited to, data format requirements, prerequisite operations, invocation formats, and the like, and any combination thereof that are particular to each tool. Additional tools and operations may also be easily built into the workflow system as add-on modules. Such “add-on modules,” comprise tools which are apart from the various workflows themselves, and may include, for example, providing an interface, using a standard set of variables defined by the workflows described herein, for performing certain functions. These modules may also specify, for example, alternate tools that are available to implement certain base workflow functions (e.g., alignment, quantification, and the like), and/or available additional or optional steps and/or functions to the base workflow (e.g., the dashed-lined functions in FIGS. 1 and 2), and the like, and any combination thereof. Accordingly, in some embodiments, the user selects a specific tool that is already incorporated into the workflow for performing a certain analytical function or may specify one or more additional, add-on modules (or tools) or particular combination of tools (existing or additional) to the workflow for analysis, without departing from the scope of the present disclosure.

The RNA-Seq workflows of the present disclosure for performing differential expression analysis additionally feature adaptive data specification. That is, in some embodiments, the RNA-Seq read data may be structured based on user rules for identifying specific datasets for analysis (e.g., FASTA files). Such user-specified directives (e.g., rules or instructions) for identification of the desired datasets for analysis may be determined, for example, at runtime based on a file naming construction identified by and specific to the user. Alternatively, the file naming construction for use in identifying the dataset(s) for analysis by the workflow described herein may be conventional or otherwise used by more than one user. In such a way, users may easily identify specific datasets for analysis, and maintain those datasets separate from others or share certain datasets based on file naming conventions, if appropriate. In both instances, the user-specified directives allow the workflow to automatically identify the dataset of interest. From these directives, the workflow determines how to combine each replicate and/or condition into appropriate groupings (e.g., combination pairs) and how many differential expression analyses must be computed based on such groupings. As an example, the user dataset identification rules may specify the different dataset elements via what are known as “wildcard” or “regular” expressions, as known to those of skill in the art. For instance, a data file format specification of “EM*fastq*” will match any file whose filename starts with “EM” and includes “fastq” somewhere in the name or extension. In other words, the “*” character is an indication to “match any character zero or more times.” In this or other instances, the implementation may also allow explicit enumeration of all of the data elements/filenames to consider by the workflow.

The RNA-Seq workflow of the present disclosure is designed to abstract its computational aspects. That is, the user of the workflows described herein, is abstracted (i.e., unaware or blinded) from the computational aspects of the differential expression analysis, which are managed by the workflow. Moreover, changes in computing devices and/or parallelization management can be adapted using the workflow and remain transparent to the user. It is this transparency, as well as the computation standardization of the workflows described herein, among other aspects of the workflows described herein (e.g., faster analysis results (such as days rather than traditional weeks or months), ease of add-on modules, and the like) that contribute to its user-friendly nature.

In one or more aspects, the workflow system may allocate and manage the parallelization of different computational elements, as further described below. Such parallelization allows each RNA-Seq read sample (i.e., pertaining to a particular biological sample) to be processed independently of all other sample RNA-Seq reads for certain analyses, such as alignment and quantification of expression, and thereafter to be compared to each and every other sample in the identified dataset for differential expression analysis (e.g., see FIGS. 1 and 2). The parallelization further establishes certain ordering for performing the steps of the RNA-Seq workflows described herein (i.e., which steps are performed before or after other steps). In some embodiments, this parallelization may be achieved using at least one data processing unit (e.g., comprising at least one processor and memory), as described below, according to an automatically generated dependency graph. A “dependency graph,” as used herein, and grammatical variants thereof, is used to establish at least partial ordering among the steps of the workflows described herein. Additionally, the parallelization (e.g., according to an automatically generated dependency graph) may translate to ease of use, with users able to merely specify datasets and specific workflow tools out the outset (e.g., at runtime) via an analysis configuration file and user interface. Thereafter, the RNA-Seq workflow of the present disclosure performs the requisite steps for ultimate differential expression analysis of combination pairs without additional user intervention, and as previously specified by the user.

Parallelization may occur at various levels of the workflow (e.g., along the flowchart of FIGS. 1 and 2). For examples, parallelization may occur at the particular tool level (i.e., each functional tool is parallelized across multiple processing units for the specified input data/files), or at the data level (i.e., each data operation is performed in parallel for mutually exclusive sets of data files), and any combination thereof. The role of the dependency graph described above is to determine aspects of the data analysis can take place in parallel based on various dependencies, for example. That is, in some embodiments, certain operations of the workflow depend from prior operations, such that certain operations can only proceed upon performing prerequisite operations (e.g., such dependency is illustrated by the lines from top to bottom in FIGS. 1 and 2, the arrows being from prerequisite to subsequent operations). Accordingly, for example, for a given subset of one or more data files, the pre-processing, aligning, sorting, quantifying of gene expression, and normalization operations are sequential because each operation requires the results of the previous, and within-tool parallelization may additionally be performed.

In one or more embodiments, the RNA-Seq workflows for performing differential expression analysis described herein may be executed by running one or more configuration files (i.e., a file used to configure the parameters and initial settings of the RNA-Seq workflow). The configuration files may be executed using a computing device (or a processor-based device) that includes one or more processors, memory coupled to the one or more processors, and instructions provided to or otherwise stored in the memory and executable by the processor (collectively a “processing unit”). Any one or more suitable processor-based device(s) may be utilized for implementing all or a portion of the various RNA-Seq differential expression analysis workflow embodiments described herein. Such processor-based devices may include, but are not limited to, personal computers, networks personal computers, laptop computers, computer workstations, mobile devices, multi-processor servers or workstations with (or without) shared memory, high performance computers, and the like. The devices may be further connected via a network that allows them to communicate to exchange data or share tasks, such as in the form of a “computer cluster” or simply “cluster.” Moreover, embodiments may be implemented on application specific integrated circuits (ASICs) or very large scale integrated (VLSI) circuits.

The memory for storing instructions for performing the workflows and configuration files for execution of the workflows described herein may be any non-transitory, computer-readable medium, tangible machine-readable medium, or the like. Such memory may include, but is not limited to, any tangible storage that participates in providing instructions to one or more processors including non-volatile and volatile media. Examples of suitable memory may include, but are not limited to, a floppy disk, a flexible disk, a hard disk, magnetic tape, or any other magnetic medium, magneto-optical medium, a CD-ROM, and any other optical medium, a RAM, a PROM, an EPROM, a FLASH-EPROM, a solid state medium like holographic memory, a memory card, or any other memory chip or cartridge, or any other physical medium from which a computer can read. When the memory (e.g., computer-readable media) is configured as a database, it is to be understood that the database may be any type of database, such as relational, hierarchical, object-oriented, and/or the like. Accordingly, exemplary embodiments of the present techniques may be considered to include a tangible storage medium or tangible distribution medium and recognized equivalents and successor media.

In one or more aspects of the present disclosure, a method is provided of using at least one data processing unit comprising at least one processor and memory coupled to the at least one processor, the memory operative to store instructions that, when executed by the processor, cause the processor to perform the automated differential analysis expression of RNA-Seq data workflow(s) described herein. Further, according to one or more aspects of the present disclosure, a system is provided comprising at least one data processing unit including at least one processor and memory coupled to the at least one processor, the memory operative to store instructions that, when executed by the processor, cause the processor to perform the automated differential expression analysis of RNA-Seq data workflow(s) described herein.

The computing device may be in electrical communication with one or more displays or graphical user interfaces, which may be used interchangeably herein, electrically coupled to one or more data processing units. The display may permit user input for initial specification of a particular workflow according to the embodiments described herein, for modification of the workflow and selection of desired tools of interest, and the like, and any combination thereof. The display may be any of a computer screen allowing the user to input certain information for performing the workflow (e.g., a keyboard or other buttons or knobs), a touchscreen, such as for entering information and commands using a finger or stylus, and the like, and any combination thereof. The display may further be configured for displaying various data or information related to the RNA-Seq read differential expression analysis performed according to the workflows described herein. For example, in one embodiment, the RNA-Seq nucleotide data may be displayed, the transcriptome loci that the RNA-Seq data aligns against, the counts of the RNA-Seq data related to one or more loci of the transcriptome, and any other data or graphical representations thereof associated with the performance of the workflows. In some embodiments, for example, the display may be configured to display a graphical representation of the differential gene expression between each combination pair of experimental conditions of a plurality of RNA-Seq reads. For example, the graphical representation may be a chart (e.g., bar chart, pie chart, line chart, and the like), a data table, or matrix graphically displaying the difference in gene expression of one or more particular genes among combination pairs.

The instructions for use in the methods and systems described herein are used to execute the RNA-Seq data workflows of the present disclosure, including identifying a plurality of RNA-Seq reads from genomic samples, where the plurality of RNA-Seq reads have been subjected to at least one experimental condition (e.g., a combination pair of experimental conditions). For example, the plurality of RNA-Seq reads may come from a plurality of genomic samples from biological samples (e.g., microorganisms), where each biological sample is subjected to at least one experimental condition. That is, the biological samples may be subjected to the exact same experimental condition (i.e., in replicate) or variations of experimental conditions based on the same variable (e.g., variations in concentration of the same drug or other external agent). As such, a plurality of RNA-Seq reads is obtained for each biological sample subjected to each experimental condition, whether such conditions are identical or variants thereof. For use in one or more embodiment workflows described herein, it is preferred that the biological samples are of the same biological specimen (e.g., species), such that the variation in differential expression of the RNA-Seq reads is not dependent upon the particular biological specimen across samples, except for natural variation. Nevertheless, the workflows described herein are not limited to only single biological specimen analysis and may be used to analyze samples of cultures with multiple biological species and RNA-Seq reads thereof, without departing from the scope of the present disclosure.

Each such biological sample will yield a genomic sample having multiple RNA-Seq reads for evaluation. In some embodiments, one or more tools may be incorporated in the workflows of the present disclosure to evaluate the RNA-Seq reads associated with each genomic sample, each genomic sample representing the influence of at least one experimental condition. The workflows of the present disclosure may thereafter be used to identify the plurality of RNA-Seq reads from a plurality of genomic samples each subjected to at least one experimental condition (including control conditions), align the plurality of RNA-Seq reads to a transcriptome that is complementary to the biological specimen(s) from which the genomic samples were derived (i.e., the same species as the genomic samples or multiple species if applicable), quantify gene expression for the RNA-Seq reads, and quantify differential expression in the plurality of RNA-Seq reads between combination pairs of the experimental condition. Other tools may be used for various additional quality control, evaluation, and analysis as part of the workflows described herein, without departing from the scope of the present disclosure.

Referring to FIG. 1, illustrated is a schematic flowchart demonstrating one or more aspects of the parallel analysis (e.g., identifying (labeled as “input data”), pre-processing, aligning, quantifying gene expression, and quantifying differential gene expression) and combination pairing of the RNA-Seq workflows of the present disclosure. As shown, Species Q (e.g., a particular microorganism species) has been treated with Conditions A, B, and C (e.g., concentrations of a particular drug). The RNA-Seq reads of Conditions A, B, and C of Species Q are input data for use in an RNA-Seq workflow according to one or more embodiments of the present disclosure. Such data may be in the form of stored files (e.g., FASTA files) that are identifiable by the workflow according to a particular default naming convention, or alternatively as specified by a user (e.g., using a user display or interface). The naming convention allows the workflow to identify the desired data set for comparison, as well as the specific conditions for each genomic sample (and replicates, as described below). Once identified, a default workflow may be initiated or a user-specified workflow may be initiated to perform at least alignment, quantification of gene expression, and quantification of differential gene expression between each combination pair of experimental conditions.

As shown in FIG. 1, in some embodiments, the identified RNA-Seq data from genomic samples of Species Q subjected to experimental Conditions A, B, and C may be optionally pre-processed (shown in phantom) (e.g., with the data processing unit). As used herein, the term “pre-processed” or “pre-processing,” and grammatical variants thereof, refers to any method aimed at reducing potential errors associated with sequencing of nucleic acids. Pre-processing may be used as a form of quality control to enhance the RNA-Seq read dataset used for downstream analysis (e.g., alignment, gene quantification, and differential expression, among others) performed using the workflow of the present disclosure.

Examples of pre-processing include, but are not limited to, removing barcoded sequences used to identify each sample (e.g., nucleic acid sequences for labeling each RNA-Seq read for indexing or library formation purposes), trimming extremities of RNA-Seq reads to reduce potential sources of differential gene expression analysis error, trimming RNA-Seq reads having a low sequence quality score, any other quality control measure, and any combination thereof. The workflow and/or a user may assign a sequence quality score threshold. Typically, RNA-Seq read quality decreases towards the 3′ ends and when a certain low threshold is met, the low quality bases may be removed to improve alignment operations. In one or more examples, for instance, the user may specify the quality score, such as a minimum average quality score of 18 nucleotide bases over a window of 20 nucleotide bases, regardless of the location in the RNA-Seq read. Other quality measures may also be employed during pre-processing, without departing from the scope of the present disclosure.

With continued reference to FIG. 1, after optionally pre-processing the RNA-Seq reads from the various Conditions A, B, and C of genomic samples of Species Q, the RNA-Seq reads are aligned to a transcriptome for the genomic samples. That is, the transcriptome is aligned de novo or based on a reference transcriptome of the same biological species used to produce the RNA-Seq reads (e.g., Species Q in FIG. 1). Multiple transcriptomes can also be combined to extend the analysis to cultures with multiple species, as stated hereinabove. The transcriptome may be a complete or partial set of RNA transcribed from the particular genome, and is preferably a complete set. When the transcriptome is a reference transcriptome, it may be stored within the memory and accessible by the computing device described herein.

As used herein, the term “alignment,” and grammatical variants thereof, refers to the process of locating the position of a genomic sample RNA-Seq read to a location on a transcriptome. Alignment informs which portions of the transcriptome (e.g., which genes of the biological sample) are expressed and transcribed, or up- or down-regulated, upon exposure to a particular experimental condition(s). Alignment may be performed de novo or by comparison to a reference transcriptome. The alignment may include aligning short portions of the RNA-Seq reads to the transcriptome and thereafter using dynamic programming to optimize the alignment. The workflow described herein may allow a user to select one or more tools for performing alignment, which may be selected separately for each run, for example, or certain tools may be default selected. Such tools may include, but are not limited to, Bowtie, MAPQ, SOAP, HISAT, TopHat, Subread, STAR, Sailfish, Kallisto, GMAP, BWA, Salmon, and the like, and any combination thereof.

Referring again to FIG. 1, subsequent to alignment, the workflows described herein may quantify gene expression for the plurality of RNA-Seq reads. The quantification of gene expression for the various RNA-Seq reads for each biological sample may be achieved by any suitable method for counting the number of RNA-Seq reads per genome sample that aligned to each locus (e.g., gene or exon) of the transcriptome during alignment. The counts represent the type and amount of transcribed (and thus translated) RNA in each genome sample under the particular experimental condition to which it was subjected. The quantification of the expression may be achieved by one or more tools used and accessed through the workflow of the present disclosure, which may be default in the workflow and/or user selected. Such tools may include, but are not limited to, HTSeq, FeatureCounts, Rcount, maxcounts, FIXSEQ, Cuffquant, and the like, and any combination thereof.

The workflow of the present disclosure, as depicted in FIG. 1, performs automated quantification of differential gene expression by comparing the gene expression of each of the RNA-Seq reads subjected to Condition A, B, and C—thus, forming combination pairs for the experimental Conditions A&B, A&C, and B&C. Unlike previous analysis tools, therefore, each combination pair is evaluated to determine differential gene expression in order to assess the effect of the experimental conditions between the two samples. This differential expression comparison for each combination pair allows large datasets to be seamlessly evaluated in a single workflow step without any additional pairing steps and without risk of omitting one or more of the combination pair comparisons. The differential gene expression between each combination pair indicates whether one or more particular genes are expressed in the presence or absence of a particular experimental condition or are expressed in a different amount in the presence of a particular experimental condition. The quantification of the differential expression may be achieved by one or more tools used and accessed through the workflow of the present disclosure, which may be default in the workflow and/or user selected. Such tools may include, but are not limited to, Cuffdiff, DESeq, edgeR, and any combination thereof.

Notably, FIG. 1 demonstrates the automatic dependency graph that may be generated using one or more data processing units of the present disclosure according to the workflow described herein. Each of the identifying, pre-processing, aligning, quantifying of gene expression, and differential expression analysis are performed in parallel—independently and in a set order—for each experimental condition. Such parallelization may be performed on a single data processing unit or a plurality of data processing units. Thereafter, each of the experimental conditions is paired with one other experimental condition in order to obtain quantification of differential expression for every possible combination pair in the identified dataset.

Referring now to FIG. 2, illustrated is a schematic flowchart demonstrating one or more aspects of the parallel analysis (e.g., identifying, (labeled as “input data”), pre-processing, aligning, sorting, quantifying gene expression, normalizing, quantifying differential gene expression, and differential expression analysis) and combination pairing of the RNA-Seq workflows of the present disclosure. As shown, Species Q (e.g., a particular microorganism species) has been treated with Conditions A, B, and C (e.g., concentrations of a particular drug), and each Condition has been tested with replicates (“replicate 1” “replicate 2” and, for Condition B, “replicate 3”). The RNA-Seq reads of each replicate of Conditions A, B, and C of Species Q are input data for use in an RNA-Seq workflow according to one or more embodiments of the present disclosure.

Notably, as with FIG. 1, FIG. 2 also demonstrates the automatic dependency graph that may be generated using one or more data processing units of the present disclosure according to the workflow described herein. Each of the identifying, pre-processing, aligning, sorting, quantifying of gene expression, normalization, and differential expression analysis are performed in parallel—independently and in a set order for each experimental condition and each replicate. Such parallelization may be performed on a single data processing unit or a plurality of data processing units. Thereafter, each of the normalized experimental conditions is paired with one other normalized experimental condition in order to obtain quantification of differential expression for every possible combination pair in the identified dataset (i.e., Conditions A&B, A&C, and B&C as shown in FIG. 2).

For brevity, like workflow steps and processes described above with reference to FIG. 1 will not be repeated with reference to FIG. 2. That is, the identifying (input data), pre-processing, aligning, quantification of gene expression, and quantification of differential gene expression described in FIG. 1 is equally applicable to FIG. 2, without limitation.

As shown in FIG. 2, optionally, the aligned RNA-Seq reads may further undergo sorting (i.e., after at least aligning, the workflow may be used to sort the aligned RNA-Seq reads using at least one data processing unit). As used herein, the term “sorting,” and grammatical variants thereof, refers to the reordering of a plurality of aligned RNA-Seq reads such that they are ordered according to the alignment location within the transcriptome. Sorting may facilitate performance of subsequent operations, especially with regard to the quantification of RNA-Seq data with paired reads. The sorting of the aligned RNA-Seq reads may be achieved by one or more tools used and accessed through the workflow of the present disclosure, which may be default in the workflow and/or user selected. Such tools may include, but are not limited to, samtools, Pysam, Picard, and the like, and any combination thereof. The term “sam” (or in some instances “bam”) refers to a particular file format for storing RNA-seq read alignments.

With continued reference to FIG. 2, the replicates for each of Conditions A, B, and C may be optionally normalized prior to differential expression analysis of each of the combination pairs. As used herein, the term “normalized,” and grammatical variants thereof (e.g., normalizing, normalization, and the like), refers to a transformation of quantified gene expression levels to account for potential systematic bias and to permit accurate comparison of relevant differential expression levels. Typically, the transformation may be achieved using statistical analysis of quantified expression levels compared to a reference value (e.g., variance accounting for sampling error, total sampling output evaluation, gene length evaluation, and the like). Systematic bias may be the result of human error, testing equipment error, natural variation among otherwise identical biological specimens, and the like, and any combination thereof. As shown in FIG. 2, each of the replicates is normalized and a single quantification for gene expression is determined for subsequent quantification of differential expression between each combination pair.

It is to be appreciated, however, that RNA-Seq read genome samples may be normalized regardless of whether there are duplicates (e.g., against a reference), without departing from the scope of the present disclosure. Moreover, it is to be appreciated that the workflows of the present disclosure may normalize (or opt not to normalize) one or more replicates and thereafter permit comparison of combination pairs including each of the replicate RNA-Seq data (e.g., rather than averaging the replicates, for example), without departing from the scope of the present disclosure. In such instances, with reference to FIG. 2, the combination pairs would include A1&B1, A2&B2, A1&C1, A1&C2, and so on.

The normalizing of the quantified RNA-Seq reads may be achieved by one or more tools used and accessed through the workflow of the present disclosure, which may be default in the workflow and/or user selected. Such tools may include, but are not limited to, cuffnorm, or implicitly built-in within cuffdiff, DESeq, or edgeR, and the like, and any combination thereof.

In some embodiments, accordingly, the methods and systems described herein include automated differential expression analysis of a plurality of RNA-Seq reads from genome samples. The plurality of RNA-Seq reads may comprise two or more replicates for each genome sample, such as to account for natural variation or variation introduced during sampling and/or testing. Such plurality of RNA-Seq reads may be subjected to different experimental conditions, including different external agent exposure, different concentrations of such external agent exposure, the absence of such external agent exposure, and the like and any combination thereof. As discussed above, the plurality of RNA-Seq reads may be in the millions for each genome sample, or at least two, or at least three, or more RNA-Seq reads to permit differential expression analysis according to one or more workflows of the present disclosure.

The workflows may be user specified and modifiable, including identification of specific RNA-Seq datasets for analysis and selection of one or more tools for each step in the workflow. In some embodiments, at least one data processing unit of a computing device may receive user specified instructions for defining one or more parameters (e.g., datasets, tools, ordered steps, and the like), such as through a display configured for manipulation (i.e., data input) by a user. As previously provided, the one or more parameters specified by the user may include, but are not limited to, a location of one or more user files for identifying a plurality of RNA-Seq reads from genomic samples, the identification performed by at least one data processing unit.

A particular advantage of the present disclosure includes the parallelization of data according to an automatically generated dependency graph, as described above. In one or more embodiments, for example, at least two, or more, or all of the selected operations of the workflow (e.g., pre-processing, aligning, sorting, quantification of gene expression, normalizing, and differential expression analysis) are parallelized according to a dependency graph automatically generated by the workflows of the present disclosure, as described hereinabove. The parallelization may be performed using one or a plurality of data processing units (e.g., across a plurality of data processing units, as made available in a computer cluster), without departing from the scope of the present disclosure. Thereafter, the parallelized (and independently ordered) operations are combined into combination pairs for differential expression analysis. Each path from top to bottom in FIGS. 1 and 2 are independent operations, except for their dependency in the direction of the arrows as discussed above, and one, some, or all may be parallelized, according to the embodiments of the present disclosure.

In one or more embodiments of the present disclosure, the workflows described herein may be further streamlined such that upon meeting a certain threshold value, a subsequent operation in the workflow will proceed automatically. For example, a threshold value may be assigned to a plurality of RNA-Seq reads based on the quantification of gene expression and/or the normalization of gene expression of the plurality of RNA-Seq reads. In some embodiments, for example, an expression threshold value may be assigned to the RNA-Seq read data before or during quantifying gene expression thereof. As gene quantification proceeds, the workflow may automatically trigger quantification of differential gene expression of combination pairs once the expression threshold value is met or exceeded. In other embodiments, whether or not an expression threshold value is set, a normalized threshold may be assigned to the RNA-Seq read data before or during (optional) normalizing thereof. As normalization proceeds, the workflow may automatically trigger quantification of differential gene expression of combination pairs once the normalized threshold value is met or exceeded. In each instance, the expression and/or normalized threshold value may be a value of fold changes in expression between 2 conditions or p value below a certain threshold (e.g., 0.05). In other instances, the dependency upon a prerequisite operation being completed may be the threshold value, such as completion of alignment prior to proceeding to quantification, as described hereinabove.

Embodiments disclosed herein include:

### Embodiment A

A method comprising: using at least one data processing unit comprising at least one processor and memory coupled to the at least one processor, the memory operative to store instructions that, when executed by the processor, cause the processor to perform an automated differential expression analysis of RNA-Sequencing (RNA-Seq) data workflow by: identifying a plurality of RNA-Seq reads from genomic samples, the plurality of genomic samples having been subjected to at least one experimental condition; aligning the plurality of RNA-Seq reads to a transcriptome for the genomic samples; quantifying gene expression for the plurality of RNA-Seq reads; and quantifying differential gene expression in the plurality of RNA-Seq reads between a combination pair of the experimental condition.

Embodiments A may have one or more of the following additional elements in any combination:

Element A1: Wherein a display is coupled to the data processing unit, and further comprising displaying, with the data processing unit, a graphical representation of the differential gene expression between each combination pair of experimental conditions of the plurality of RNA-Seq reads on the display.

Element A2: Further comprising pre-processing, with the data processing unit, the RNA-Seq reads before aligning the plurality of RNA-Seq reads to the transcriptome.

Element A3: Further comprising: assigning an expression threshold value to the plurality of RNA-Seq reads before or during quantifying the gene expression of the plurality of RNA-Seq reads, and proceeding with quantifying the differential gene expression of the plurality of RNA-Seq reads when the expression threshold value is met exceeded.

Element A4: Further comprising sorting, with the data processing unit, the RNA-Seq reads after aligning and before quantifying gene expression for the plurality of RNA-Seq reads.

Element A5: Further comprising normalizing, with the data processing unit, the quantified gene expression for the plurality of RNA-Seq reads before quantifying differential gene expression between each combination pair of the plurality of RNA-Seq reads.

Element A6: Further comprising normalizing, with the data processing unit, the quantified gene expression for the plurality of RNA-Seq reads before quantifying differential gene expression between each combination pair of the plurality of RNA-Seq reads; and further comprising assigning a normalized threshold value to the plurality of RNA-Seq reads before or during normalizing the quantified gene expression of the plurality of RNA-Seq reads, and proceeding with quantifying the differential gene expression of the plurality of RNA-Seq reads when the normalized threshold value is met exceeded.

Element A7: Wherein the plurality of RNA-Seq reads comprises at least two replicates subjected to the same experimental condition.

Element A8: Wherein the plurality of RNA-Seq reads were subjected to different experimental conditions.

Element A9: Further comprising providing, with the data processing unit, at least two gene expression option tools for quantifying gene expression for the plurality of RNA-Seq reads.

Element A10: Further comprising providing, with the data processing unit, at least two differential gene expression option tools for quantifying differential gene expression between each combination pair of experimental conditions of the plurality of RNA-Seq reads.

Element A11: Further comprising receiving user specified instructions for defining parameters of the workflow.

Element A12: Further comprising receiving user specified instructions for defining parameters of the workflow, and wherein the parameters comprise a location of one or more user files for identifying the plurality of RNA-Seq reads from the genomic samples by the data processing unit.

Element A13: Wherein at least two operations of the workflow are parallelized according to an automatically generated dependency graph.

Element A14: Wherein at least two operations of the workflow are parallelized according to an automatically generated dependency graph, and further comprising a plurality of data processing units, and wherein the operations of the workflow are parallelized across the plurality of data processing units.

Element A14: Further comprising receiving user input defining the automated differential expression analysis of RNA-Sequencing data workflow comprising one or more user specified directives.

By way of non-limiting example, exemplary combinations applicable to A include: A1 and A2; A1 and A3; A1 and A4; A1 and A5; A1 and A6; A1 and A7; A1 and A8; A1 and A9; A1 and A10; A1 and A11; A1 and A12; A1 and A13; A1 and A14; A2 and A3; A2 and A4; A2 and A5; A2 and A6; A2 and A7; A2 and A8; A2 and A9; A2 and A10; A2 and A11; A2 and A12; A2 and A13; A2 and A14; A3 and A4; A3 and A5; A3 and A6; A3 and A7; A3 and A8; A3 and A9; A3 and A10; A3 and A11; A3 and A12; A3 and A13; A3 and A14; A4 and A5; A4 and A6; A4 and A7; A4 and A8; A4 and A9; A4 and A10; A2 and A11; A4 and A12; A4 and A13; A4 and A14; A5 and A6; A5 and A7; A5 and A8; A5 and A9; A5 and A10; A5 and A11; A5 and A12; A5 and A13; A5 and A14; A6 and A7; A6 and A8; A6 and A9; A6 and A10; A6 and A11; A6 and A12; A6 and A13; A6 and A14; A7 and A8; A7 and A9; A7 and A10; A7 and A11; A7 and A12; A7 and A13; A7 and A14; A8 and A9; A8 and A10; A8 and A11; A8 and A12; A8 and A13; A8 and A14; A9 and A10; A9 and A11; A9 and A12; A9 and A13; A9 and A14; A10 and A11; A10 and A12; A10 and A13; A10 and A14; A11 and A12; A11 and A13; A11 and A14; A12 and A13; A12 and A14; A13 and A14; and any non-limiting combination of one, more, or all of A1, A2, A3, A4, A5, A6, A7, A8, A9, A10, A11, A12, A13, and/or A14.

### Embodiment B

A system comprising: at least one data processing unit comprising at least one processor and memory coupled to the at least one processor, the memory operative to store instructions that, when executed by the processor, cause the processor to perform an automated differential expression analysis of RNA-Sequencing (RNA-Seq) data workflow, the workflow configured to: identify a plurality of RNA-Seq reads from genomic samples, the plurality of genomic samples having been subjected to at least one experimental condition; align the plurality of RNA-Seq reads to a transcriptome for the genomic samples; quantify gene expression for the plurality of RNA-Seq reads; and quantify differential gene expression in the plurality of RNA-Seq reads between a combination pair of the experimental condition.

Embodiments B may have one or more of the following additional elements in any combination:

Element B1: Wherein a display is coupled to the data processing unit and configured to display a graphical representation of the differential gene expression between each combination pair of experimental conditions of the plurality of RNA-Seq reads.

Element B2: Wherein the workflow is further configured to pre-process the RNA-Seq reads before aligning the plurality of RNA-Seq reads to the transcriptome.

Element B3: Wherein the workflow is further configured to assign an expression threshold value to the plurality of RNA-Seq reads before or during quantifying the gene expression of the plurality of RNA-Seq reads, and proceed with quantifying the differential gene expression of the plurality of RNA-Seq reads when the expression threshold value is met exceeded.

Element B4: Wherein the workflow is further configured to sort the RNA-Seq reads after aligning and before quantifying gene expression for the plurality of RNA-Seq reads.

Element B5: Further comprising normalizing, with the data processing unit, the quantified gene expression for the plurality of RNA-Seq reads before quantifying differential gene expression between each combination pair of the plurality of RNA-Seq reads.

Element B6: Further comprising normalizing, with the data processing unit, the quantified gene expression for the plurality of RNA-Seq reads before quantifying differential gene expression between each combination pair of the plurality of RNA-Seq reads; and wherein the workflow is further configured to assign a normalized threshold value to the plurality of RNA-Seq reads before or during normalizing the quantified gene expression of the plurality of RNA-Seq reads, and proceed with quantifying the differential gene expression of the plurality of RNA-Seq reads when the normalized threshold value is met exceeded.

Element B7: Wherein the plurality of RNA-Seq reads comprises at least two replicates subjected to the same experimental condition.

Element B8: Wherein the plurality of RNA-Seq reads were subjected to different experimental conditions.

Element B9: Wherein the workflow is further configured to provide at least two gene expression option tools for quantifying gene expression for the plurality of RNA-Seq reads.

Element B10: Wherein the workflow is further configured to provide at least two differential gene expression option tools for quantifying differential gene expression between each combination pair of experimental conditions of the plurality of RNA-Seq reads.

Element B11: Wherein the workflow is further configured to receive user specified instructions for defining parameters of the workflow.

Element B12: Wherein the workflow is further configured to receive user specified instructions for defining parameters of the workflow, and wherein the parameters comprise a location of one or more user files for identifying the plurality of RNA-Seq reads from the genomic samples by the data processing unit.

Element B13: Wherein at least two operations of the workflow are parallelized according to an automatically generated dependency graph.

Element B14: Wherein at least two operations of the workflow are parallelized according to an automatically generated dependency graph; and further comprising a plurality of data processing units, and wherein the operations of the workflow are parallelized across the plurality of data processing units.

Element B15: Wherein the workflow is configured to receive user input defining the automated differential expression analysis of RNA-Sequencing data workflow comprising one or more user specified directives.

By way of non-limiting example, exemplary combinations applicable to B include: B1 and B2; B1 and B3; B1 and B4; B1 and B5; B1 and B6; B1 and B7; B1 and B8; B1 and B9; B1 and B10; B1 and B11; B1 and B12; B1 and B13; B1 and B14; B1 and B15; B2 and B3; B2 and B4; B2 and B5; B2 and B6; B2 and B7; B2 and B8; B2 and B9; B2 and B10; B2 and B11; B2 and B12; B2 and B13; B2 and B14; B2 and B15; B3 and B4; B3 and B5; B3 and B6; B3 and B7; B3 and B8; B3 and B9; B3 and B10; B3 and B11; B3 and B12; B3 and B13; B3 and B14; B3 and B15; B4 and B5; B4 and B6; B4 and B7; B4 and B8; B4 and B9; B4 and B10; B2 and B11; B4 and B12; B4 and B13; B4 and B14; B4 and B15; B5 and B6; B5 and B7; B5 and B8; B5 and B9; B5 and B10; B5 and B11; B5 and B12; B5 and B13; B5 and B14; B5 and B15; B6 and B7; B6 and B8; B6 and B9; B6 and B10; B6 and B11; B6 and B12; B6 and B13; B6 and B14; B6 and B15; B7 and B8; B7 and B9; B7 and B10; B7 and B11; B7 and B12; B7 and B13; B7 and B14; B7 and B15; B8 and B9; B8 and B10; B8 and B11; B8 and B12; B8 and B13; B8 and B14; B8 and B15; B9 and B10; B9 and B11; B9 and B12; B9 and B13; B9 and B14; B9 and B15; B10 and B11; B10 and B12; B10 and B13; B10 and B14; B10 and B15; B11 and B12; B11 and B13; B11 and B14; B11 and B15; B12 and B13; B12 and B14; B12 and B15; B13 and B14; B13 and B15; B14 and B15; and any non-limiting combination of one, more, or all of B1, B2, B3, B4, B5, B6, B7, B8, B9, B10, B11, B12, B13, B14; and/or B15.

To facilitate a better understanding of the embodiments of the present invention, the following examples of preferred or representative embodiments are given. In no way should the following examples be read to limit, or to define, the scope of the disclosure.

### EXAMPLES

### Example 1

A non-limiting example of configuration file code for data specification for the RNA-Seq read data workflows according to one or more aspects of the embodiments described herein is provided below in Table 1. The data specification code may be used to determine the location of desired RNA-Seq read datasets, conditions thereof, and replicates thereof used in the workflow operations that proceed thereafter to effectively analyze differential gene expression of precise combination pairs. The data specification may be requested from a user and, thus, user specified. In other embodiments, the data specification may be integrated into the workflow, such as a default data specification protocol. It is to be appreciated that one or more aspects of the workflow configuration file may be modified or otherwise adapted for specific user preferences, without departing from the scope of the present disclosure. The lines started by “#” in Table 1 provide additional details.

TABLE 1
# Location of the genome file (e.g., a FASTA file)
genomeFile=“genome.fasta”
# Location of the transcript annotation file (e.g., GFF or GTF file)
annotationFile=“annotation.gff”
# Directory to access/store the genome index files.
genomeIdxDir=“genome_indices”
# List of directories, one per condition (explicitly or via a name format specification)
conditionDirs=( cond*)
# Specifies a format to identify the files that contain the reads (e.g., simply “*”, but more
specificity may be preferred to aid in differentiating data files from other file types present in
the directories for the conditions; in some instances, regex through grep by writing “* | grep
<args>” may be used; leading spaces in the string should not be used)
dataFileFmt=“EM*fastq*”
# Command to determine a filename identifier prefix that is unique to each replicate (and also
common to all data files of that replicate); this identifier may be used as the prefix for all
replicate analysis files
identifyReplicateFilesIdentifier=“cut -d‘_’ -f1”
# Specify whether the RNA-Seq read data are paired-reads or not
pairedReads=“true”
# Specify identifiers for each set of combination pairs (or read mates); ignored for unpaired
reads
pairedReads1=“R1”
pairedReads2=“R2”

A non-limiting example of configuration file code for various workflow operations according to one or more aspects of the embodiments described herein is provided below in Table 2. The data specification code may be used to specify one or more workflow operations (e.g., alignment, gene expression quantification, differential gene expression quantification, and the like) and tools for performing such operations to effectively analyze differential gene expression of precise combination pairs. The analysis specification may be requested from a user and, thus, user specified. In other embodiments, the analysis specification may be integrated into the workflow, such as a default analysis specification protocol. It is to be appreciated that one or more aspects of the workflow configuration file may be modified or otherwise adapted for specific user preferences, without departing from the scope of the present disclosure. The “#” in Table 2 provide additional details.

TABLE 2

# Alignment algorithm to use (e.g., Bowtie, HISAT, and the like)
alignmentAlg=“HISAT”
# Specify circumstances that the user is to be notified (e.g.,
via email) of progress/issues with the workflow operations (e.g.,
ALL, BEGIN, END, or FAIL). emailUserOn=“FAIL”
# Specify whether to notify (e.g., email) user when all of the
workflow operations are complete emailUserWhenFinished=
“true”
# Specify normalization method to use prior to evaluating
differential expression (e.g., possible values may include
{‘classic-fpkm’, ‘geometric’, ‘quartile’})
normMethod=“classic-fpkm”

### Example 2

In this example, RNA-Seq data was obtained and differential expression analysis was performed according to an automated differential expression analysis workflow of the present disclosure and compared to a differential expression analysis performed commercially by a third-party vendor.

Pure cultures of Desulfovibrio vulgaris Hildenborough were grown anaerobically using a media containing lactate as electron donor and sulfate as electron acceptor. The growth media was prepared using 30 millimolar (mM) lactate, 30 mM sulphate, 8 mM MgCl₂, 20 mM NH₄Cl, 2.2 mM phosphate buffer, 0.6 mM CaCl₂, 24 mM NaCO₃, 0.02% resazurin, 0.06 mM FeCl₂, trace elements and Thauer's vitamins. The pH of the growth media was adjusted to 7.2, was sparged with 15% CO₂:N₂, and sterilized by autoclave. Sodium dithionite was added to the growth media immediately before inoculation to a final concentration of 1.5 mM.

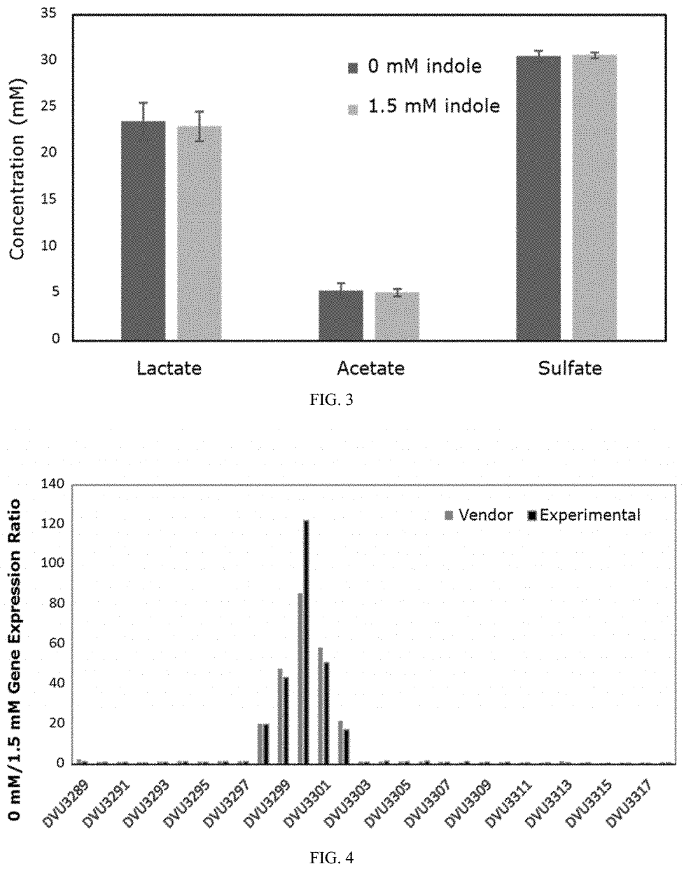

Growth media containing no indole or 1.5 mM indole was used to fill 500 milliliter (ml) serum bottles and incubated at 30° C. and 60 rpm for 18 hours in triplicate replicates. In this study, the effect of indole, a bacterial metabolite, was assessed on early planktonic cultures. Desulfovibrio vulgaris Hildenborough consumes the electron donor (lactate) and acceptor (sulfate), along with by-product (acetate) generation. The lactate, sulfate, and acetate concentration results after incubation are shown in FIG. 3. As is known to those of skill in the art, indole does not affect Desulfovibrio vulgaris Hildenborough bacterial growth at the tested concentrations.

The genetic response to indole on early exponential growth was tested using RNA-Seq. At the end of the experiment, approximately 350 ml of the planktonic cells were poured into 500 ml centrifuge tubes and centrifuged (AVANTI® JXN-26 with JA10 rotor, Beckman-Coulter, Brea, Calif., USA) at 6000 g (˜7200 rpm) for 40 min at 4° C. The resulting pellet was dissolved in 35 ml pre-chilled 1× sterile PBS and transferred into sterile 50 ml falcon tubes in ice, then centrifuged again at 3000 rpm for 40 min at 4° C. (SORVALL™ ST 40R, Thermo Fisher Scientific, Waltham, Mass., USA). After centrifugation, the remaining pellets were stored at −80° C. until ready for sequencing using HISEQ® 2500 Sequencing System, Illumina, Inc., San Diego, Calif., USA. Bioinformatic analysis was performed by a third-party vendor (comparative) and, for contrast, using the methods employing the workflows of the present disclosure (experimental). Thirty (30) genes (DVU3289-3318) were selected based on previous analysis data showing up-regulation of five (5) such genes (DVU 3298-3302) in samples without indole compared to flanking genes showing no difference between the two (2) treatments. As shown in FIG. 4, both methods capture the same magnitude in the ratios of differential expression of the targeted genes. In particular, the DVU3289-3318 cluster resulted up-regulation using the comparative commercial analysis and the workflow methodology of the present disclosure, while flanking genes did not show significant differences across the two (2) treatments, and thus the ratios were close to 1. Furthermore, the differences might be even less pronounced than shown, considering that the parameters of the invention have not been adjusted to mimic the ones used, but unknown, by the commercial vendor.

Accordingly, as shown in FIG. 4 and described herein throughout, the workflow methodology of the present disclosure not only provides streamlined and modifiable evaluation of RNA-Seq reads, but accurate and effective differential gene expression analysis of combination pairs thereof.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

1. A method comprising:
using at least one data processing unit comprising at least one processor and memory coupled to the at least one processor, the memory operative to store instructions that, when executed by the processor, cause the processor to perform an automated differential expression analysis of RNA-Sequencing (RNA-Seq) data workflow by:
identifying a plurality of RNA-Seq reads from genomic samples, the plurality of genomic samples having been subjected to at least one experimental condition;
aligning the plurality of RNA-Seq reads to a transcriptome for the genomic samples;
quantifying gene expression for the plurality of RNA-Seq reads; and
quantifying differential gene expression in the plurality of RNA-Seq reads between a combination pair of the experimental condition.

2. The method of claim 1, wherein a display is coupled to the data processing unit, and further comprising displaying, with the data processing unit, a graphical representation of the differential gene expression between each combination pair of experimental conditions of the plurality of RNA-Seq reads on the display.

3. The method of claim 1, further comprising pre-processing, with the data processing unit, the RNA-Seq reads before aligning the plurality of RNA-Seq reads to the transcriptome.

4. The method of claim 1, further comprising:
assigning an expression threshold value to the plurality of RNA-Seq reads before or during quantifying the gene expression of the plurality of RNA-Seq reads, and
proceeding with quantifying the differential gene expression of the plurality of RNA-Seq reads when the expression threshold value is met exceeded.

5. The method of claim 1, further comprising sorting, with the data processing unit, the RNA-Seq reads after aligning and before quantifying gene expression for the plurality of RNA-Seq reads.

6. The method of claim 1, further comprising normalizing, with the data processing unit, the quantified gene expression for the plurality of RNA-Seq reads before quantifying differential gene expression between each combination pair of the plurality of RNA-Seq reads.

7. The method of claim 6, further comprising:
assigning a normalized threshold value to the plurality of RNA-Seq reads before or during normalizing the quantified gene expression of the plurality of RNA-Seq reads, and
proceeding with quantifying the differential gene expression of the plurality of RNA-Seq reads when the normalized threshold value is met exceeded.

8. The method of claim 1, wherein the plurality of RNA-Seq reads comprises at least two replicates subjected to the same experimental condition.

9. The method of claim 1, wherein the plurality of RNA-Seq reads were subjected to different experimental conditions.

10. The method of claim 1, further comprising providing, with the data processing unit, at least two gene expression option tools for quantifying gene expression for the plurality of RNA-Seq reads.

11. The method of claim 1, further comprising providing, with the data processing unit, at least two differential gene expression option tools for quantifying differential gene expression between each combination pair of experimental conditions of the plurality of RNA-Seq reads.

12. The method of claim 1, further comprising receiving user specified instructions for defining parameters of the workflow.

13. The method of claim 12, wherein the parameters comprise a location of one or more user files for identifying the plurality of RNA-Seq reads from the genomic samples by the data processing unit.

14. The method of claim 1, wherein at least two operations of the workflow are parallelized according to an automatically generated dependency graph.

15. The method of claim 14, further comprising a plurality of data processing units, and wherein the operations of the workflow are parallelized across the plurality of data processing units.

16. The method of claim 1, further comprising receiving user input defining the automated differential expression analysis of RNA-Sequencing data workflow comprising one or more user specified directives.

17. A system comprising:
at least one data processing unit comprising at least one processor and memory coupled to the at least one processor, the memory operative to store instructions that, when executed by the processor, cause the processor to perform an automated differential expression analysis of RNA-Sequencing (RNA-Seq) data workflow, the workflow configured to:
identify a plurality of RNA-Seq reads from genomic samples, the plurality of genomic samples having been subjected to at least one experimental condition;
align the plurality of RNA-Seq reads to a transcriptome for the genomic samples;
quantify gene expression for the plurality of RNA-Seq reads; and
quantify differential gene expression in the plurality of RNA-Seq reads between a combination pair of the experimental condition.

18. The system of claim 17, wherein a display is coupled to the data processing unit and configured to display a graphical representation of the differential gene expression between each combination pair of experimental conditions of the plurality of RNA-Seq reads.

19. The system of claim 17, wherein the workflow is further configured to pre-process the RNA-Seq reads before aligning the plurality of RNA-Seq reads to the transcriptome.

20. The system of claim 17, wherein the workflow is further configured to assign an expression threshold value to the plurality of RNA-Seq reads before or during quantifying the gene expression of the plurality of RNA-Seq reads, and
proceed with quantifying the differential gene expression of the plurality of RNA-Seq reads when the expression threshold value is met exceeded.

21. The system of claim 17, wherein the workflow is further configured to sort the RNA-Seq reads after aligning and before quantifying gene expression for the plurality of RNA-Seq reads.

22. The system of claim 17, further comprising normalizing, with the data processing unit, the quantified gene expression for the plurality of RNA-Seq reads before quantifying differential gene expression between each combination pair of the plurality of RNA-Seq reads.

23. The system of claim 17, wherein the workflow is further configured to assign a normalized threshold value to the plurality of RNA-Seq reads before or during normalizing the quantified gene expression of the plurality of RNA-Seq reads, and
proceed with quantifying the differential gene expression of the plurality of RNA-Seq reads when the normalized threshold value is met exceeded.

24. The system of claim 17, wherein the plurality of RNA-Seq reads comprises at least two replicates subjected to the same experimental condition.

25. The system of claim 17, wherein the plurality of RNA-Seq reads were subjected to different experimental conditions.

26. The system of claim 17, wherein the workflow is further configured to provide at least two gene expression option tools for quantifying gene expression for the plurality of RNA-Seq reads.

27. The system of claim 17, wherein the workflow is further configured to provide at least two differential gene expression option tools for quantifying differential gene expression between each combination pair of experimental conditions of the plurality of RNA-Seq reads.

28. The system of claim 17, wherein the workflow is further configured to receive user specified instructions for defining parameters of the workflow.

29. The system of claim 28, wherein the parameters comprise a location of one or more user files for identifying the plurality of RNA-Seq reads from the genomic samples by the data processing unit.

30. The system of claim 17, wherein at least two operations of the workflow are parallelized according to an automatically generated dependency graph.

31. The system of claim 30, further comprising a plurality of data processing units, and wherein the operations of the workflow are parallelized across the plurality of data processing units.

32. The system of claim 17, wherein the workflow is configured to receive user input defining the automated differential expression analysis of RNA-Sequencing data workflow comprising one or more user specified directives.
